(19) United States
(12) Patent Application Publication
(10) Pub. No.: US 2008/0161890 A1
(43) Pub. Date: Jul. 3, 2008

(54) Methods, systems, and apparatuses for protecting esophageal tissue during ablation

(75) Inventor: Daniel M. Lafontaine, Plymouth, MN (US)

Correspondence Address: BROOKS, CAMERON & HUEBSCH, PLLC, 1221 NICOLLET AVENUE, SUITE 500, MINNEAPOLIS, MN, 55403, US

(73) Assignee: Boston Scientific Scimed, Inc.

(21) Appl. No.: 11/649,009

(22) Filed: Jan. 3, 2007

(51) Int. Cl. A61F 7/12; A61B 18/12; A61B 8/14

(52) U.S. Cl. 607/105; 606/41; 600/450

(57) ABSTRACT

The present disclosure is directed towards methods, systems, and apparatus for protecting esophageal tissue during ablation. An ablation system can include an esophageal catheter having a heat sink and an ablation catheter having at least one ablation element to deliver ablation energy, where heat generated by the ablation energy is absorbed by the heat sink.

### BACKGROUND

Atrial fibrillation is one of the most common heart arrhythmias, affecting millions of patients in the U.S. alone. It is a rapid, irregular heart rhythm originating in the atrial chambers of the heart, commonly causing palpitations and easy fatigability, and greatly increasing the risk of stroke. Unfortunately, its treatment remains a problem for both doctors and patients.

Tissue ablation is a promising treatment for atrial fibrillation. Tissue ablation can be performed either during an electrophysiology study or in the surgical suite. Typically, the source of a patient's heart arrhythmia is mapped, localized, and then ablated. Generally, ablation is accomplished by inserting an ablation catheter into the atrial chamber. The ablation catheter is then used to apply radiofrequency (RF) energy, electrical energy, or the ablation catheter can freeze the identified areas. This energy creates a small scar that is electrically inactive and thus incapable of generating and/or propagating heart arrhythmias.

### BRIEF DESCRIPTION OF THE DRAWINGS

FIG. 1 illustrates a side elevation view of an ablation catheter according to an embodiment of the present disclosure.

FIGS. 2A-2B illustrates a sectional view of an esophageal catheter with a heat sink according to an embodiment of the present disclosure.

FIG. 3A illustrate a system that includes an esophageal catheter with a heat sink that is in fluid communication with a portable cooler.

FIGS. 3B-3C illustrate embodiments of heat exchange elements that can be incorporated into the portable cooler shown in FIG. 3A according to the present disclosure.

FIG. 4 illustrates a cross-sectional view of a distal end of an embodiment of an esophageal catheter including a balloon according to an embodiment of the present disclosure.

FIG. 5 illustrates an esophageal catheter including two balloons according to an embodiment of the present disclosure.

FIG. 6 illustrates a cross-sectional view of an esophageal catheter according to an embodiment of the present disclosure.

FIG. 7A illustrates an esophageal catheter according to an embodiment of the present disclosure.

FIG. 7B illustrates a close-up view of a thermoelectric cooling device that can be incorporated into the esophageal catheter illustrated in FIG. 6A according to an embodiment of the present disclosure.

FIG. 8A illustrates a transesophageal echocardiogram (TEE) probe according to an embodiment of the present disclosure.

FIGS. 8B-8C illustrate a detailed view of an embodiment of the distal tip portion of the TEE probe and the heat sink in the form of a cooling jacket on the outside surface of the elongate member adjacent the distal tip portion according to the present disclosure.

FIG. 8D illustrates a cross sectional view of an embodiment of the distal tip portion of the TEE probe with a cooling jacket on the outer surface of the elongate member adjacent the distal tip portion.

FIG. 8E illustrates a cross sectional view of an embodiment of the distal tip portion of the TEE probe with a cooling jacket on the outer surface of the elongate member adjacent the distal tip portion.

FIG. 8F illustrates an embodiment of a TEE probe including a cooling jacket according to the present disclosure.

FIG. 8G illustrates an embodiment of a TEE probe including a cooling jacket according to the present disclosure.

FIG. 9 illustrates an esophageal heat sink inside an esophagus while an ablation catheter is directing energy towards a cardiac tissue site.

FIG. 10A-10B illustrate an embodiment of an esophageal stylet according to the present disclosure.

FIG. 10C illustrates an embodiment of an esophageal stylet when the esophageal stylet is inside an esophagus.

FIG. 10D illustrates an embodiment of an esophageal stylet incorporated into a transesophageal echocardiogram (TEE) probe.

FIG. 11 illustrates an embodiment of an esophageal stylet according to the present disclosure when the esophageal stylet is inside an esophagus.

FIG. 12A illustrates an esophageal catheter including an electrode according to an embodiment of the present disclosure.

FIG. 12B illustrates an embodiment of the power system in FIG. 12A according to the present disclosure.

### DETAILED DESCRIPTION

One method of treating cardiac arrhythmias is with catheter ablation therapy. Physicians can use catheters with attached electrode arrays, or other ablating devices, to create cardiac lesions that disrupt electrical pathways in cardiac tissue. In general, the goal of catheter ablation therapy is to disrupt the electrical pathways in cardiac tissue to stop the emission of and/or prevent the propagation of erratic electric impulses, thereby reducing or eliminating the occurrence of the disorder.

In some instances, however, an ablation procedure can create heat. The heat created can build up at locations outside the cardiac tissue, causing potential problems. For example, during the ablation procedure an esophageal fistula can form between the atrium and the esophagus. Atrio-esophageal fistulas are a recognized complication of intraoperative ablation of AF, with an incidence reported to be as high as one percent (1%). Atrio-esophageal fistulas are thought to be caused by an increase in temperature of the esophageal tissue, i.e., burning the esophageal tissue, during the ablation procedure.

Embodiments of the present disclosure provide for an ablation system including an ablation catheter having at least one ablation element to deliver ablation energy and an esophageal catheter having a heat sink, where at least part of the heat generated by the ablation energy is absorbed by the heat sink. As used herein, a “heat sink” refers to an object that absorbs and/or dissipates heat from another object using thermal contact (in either direct or radiant contact). In the present disclosure, the heat sink can absorb and dissipate heat from portions of the esophageal tissue using thermal contact.

Embodiments of the present disclosure also provide for a method of protecting esophageal tissue by maintaining a temperature on portions of the esophageal tissue that are being heated by ablation energy that is generated and directed towards a cardiac tissue site. In one embodiment, maintaining a temperature on portions of the esophageal tissue includes providing a heat sink to absorb thermal energy created by the ablation energy. In an alternative embodiment, maintaining a temperature on portions of the esophageal tissue includes moving the esophagus a distance away from the cardiac tissue site during the ablation procedure.

The figures herein follow a numbering convention in which the first digit or digits correspond to the drawing figure number and the remaining digits identify an element or component in the drawing. The Figures illustrated herein and the various elements of each Figure are not necessarily to scale.

As discussed herein, embodiments of the present disclosure include an ablation catheter and an esophageal catheter to be used at approximately the same time to maintain portions of the esophageal tissue at a predetermined temperature. In some embodiments, the esophageal catheter is used to cool portions of the esophageal tissue to a temperature below normal body temperature (i.e., 37° C.). In some embodiments, the esophageal catheter is used to maintain a normal body temperature (i.e., 37° C.) of the esophageal tissue while the ablation catheter is being operated. Alternatively, the esophageal catheter can be used to maintain a temperature above normal body temperature (i.e., 37° C.) that is sufficiently low to prevent the esophageal tissue from burning.

As described herein, the normal functioning of the heart relies on proper electrical impulse generation and transmission. In certain heart diseases (e.g., atrial fibrillation) proper electrical generation and transmission are disrupted or are otherwise abnormal. In order to prevent improper impulse generation and transmission from causing an undesired condition, an ablation catheter can be employed in ablation therapy.

As used herein, the terms “catheter ablation” or “ablation procedures” or “ablation therapy,” and like terms, refer to what is generally known as tissue destruction procedures. Ablation is often used in treating several medical conditions, including abnormal heart rhythms. Ablation is typically performed in a special lab called the electrophysiology (EP) laboratory. During this procedure an ablation catheter is inserted into the heart using fluoroscopy for visualization, and then an energy delivery apparatus is used to direct energy to the heart muscle. This energy either “disconnects” or “isolates” the pathway of the abnormal rhythm (depending on the type of ablation). It can also be used to disconnect the conductive pathway between the upper chambers (atria) and the lower chambers (ventricles) of the heart.

As used herein, “ablation element” refers to a functional element that delivers energy to ablate tissue, such as an electrode for delivering electrical energy. Ablation elements can be configured to deliver multiple types of energy, such as ultrasound energy and cryogenic energy, either simultaneously or serially. Electrodes can be constructed of a conductive plate, wire coil, or other means of conducting electrical energy through contacting tissue. In monopolar energy delivery, the energy is conducted from the electrode through the tissue to a ground pad, such as a conductive pad attached to the back of the patient. The high concentration of energy at the electrode site causes localized tissue ablation. In bipolar energy delivery, the energy is conducted from a first electrode to one or more separate electrodes, relatively local to the first electrode, through the tissue between the associated electrodes. Bipolar energy delivery results in more precise, shallow lesions while monopolar delivery results in deeper lesions. Both monopolar and bipolar delivery provide advantages, and the combination of their use is possible in the present disclosure. Energy can also be delivered using pulse width modulated drive signals. Energy can also be delivered in a closed loop fashion, such as a system with temperature feedback wherein the temperature modifies the type, frequency, and/or magnitude of the energy delivered.

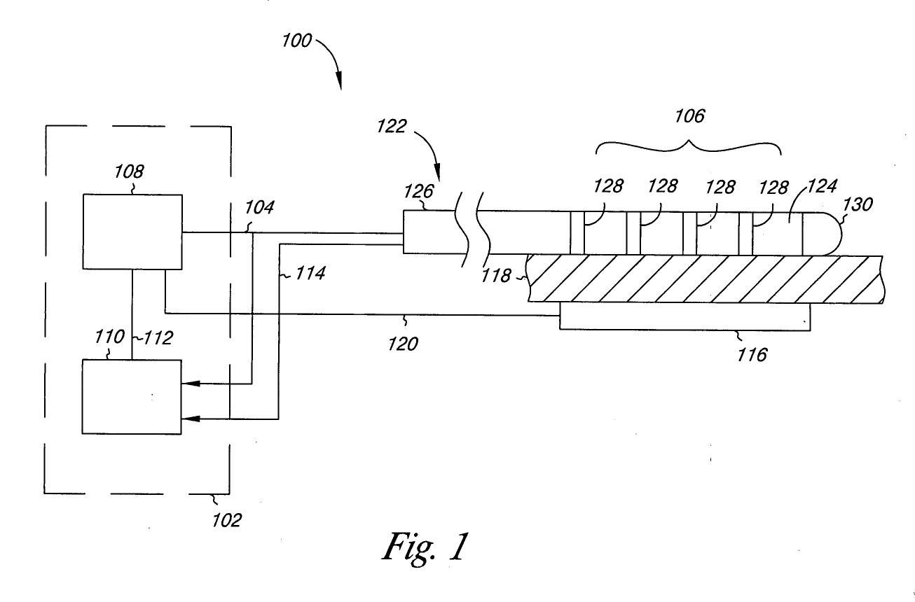

FIG. 1 illustrates a side elevation view of an ablation catheter 100 according to an embodiment of the present disclosure. Arrays of ablation elements, for example electrode arrays, may be configured in a wide variety of ways and patterns. In some embodiments, the ablation catheter 100 can include an electrode array that provides electrical energy, such as radiofrequency (RF) energy, in monopolar (unipolar), bipolar, or phase monopolar-bipolar fashion, to treat-conditions such as atrial fibrillation, supra ventricular tachycardia, atrial tachycardia, ventricular tachycardia, ventricular fibrillation, and the like. Other forms and types of energy can be delivered including, but not limited to, sound energy such as acoustic energy and ultrasound energy; electromagnetic energy such as electrical, magnetic, microwave, and radiofrequency energies; thermal energy such as heat and cryogenic energies; chemical energy such as energy generated by delivery of a drug; light energy such as infrared and visible light energies; mechanical and physical energy; radiation; and combinations thereof.

The ablation catheter 100 illustrated in FIG. 1 includes a power control system 102 that can provide power or energy 104 to an electrode device 106. In some embodiments, the power control system 102 can include a power generator 108 that can have a number of output channels through which it can provide energy 104. In addition, the operation of the power generator 108 can be controlled by a controller or processor 110 which outputs control signals 112 to the power generator 108. The controller 110 can monitor the power 104 provided by the power generator 108. In addition, in some embodiments, the controller 110 can also receive temperature signals 114 from the electrode device 106. Based on the power 104 and the temperature signals 114, the controller 110 can adjust the operation of the power generator 108.

In some embodiments, a ground pad 116 can be located proximal to the biological site 118 to be ablated opposite the site from the electrode device 106, and can be connected by a ground pad wire 120 to the power generator 108.

In some embodiments, the electrode device 106 can be part of a steerable EP catheter 122 capable of being percutaneously introduced into a biological site 118, e.g., the atrium or ventricle of the patient. In addition, the embodiment illustrated in FIG. 1 includes a distal segment 124 and a handle 126.

In some embodiments, the electrode device 106 can include band electrodes 128 arranged in a substantially linear array along the distal segment 124 of the catheter 122. The band electrodes 128 can be annular and/or semi-annular. In addition, in some embodiments, the electrode device 106 can also include a tip electrode 130.

The arrangement of the band electrodes 128 is not limited to a linear array and may take the form of other patterns. Possible electrode materials include silver, gold, chromium, aluminum, molybdenum, tungsten, nickel, platinum, and platinum/10% iridium. Other electrode materials are also possible. In addition, the electrodes 128 can be sized so that the surface area available for contact with fluid in the heart, e.g., blood, is sufficient to allow for efficient heat dissipation from the electrodes to the surrounding blood.

Embodiments of the present disclosure are not limited to the ablation catheter 100 as illustrated in FIG. 1. As will be appreciated, embodiments of the present disclosure can be used with ablation catheters taking different forms, for example, the distal segment 124 of the ablation catheter 100 can have a looped structure. In addition, in some embodiments, the ablation catheter 100 can include a cooling system to cool the ablation catheter 100 as energy is transferred from the power generator 108 to the electrodes 128.

In some embodiments, when RF energy is delivered between the electrodes 128 and the ground pad 116, there is a localized RF heating effect. This creates a well-defined, discrete lesion slightly larger than the electrodes 128 (i.e., the “damage range” for the electrode), and also causes the temperature of the tissue in contact with the electrodes 128 to rise.

In some instances, while the ablation catheter 100 is ablating cardiac tissue, the temperature of esophageal tissue can rise to the point where damage to the esophageal tissue can occur. One example of such damage is the formation of a fistula between the atria of the heart and the esophagus. As discussed herein, to reduce/minimize the occurrence of an esophageal fistula, the ablation catheter 100 can be used at approximately the same time as an esophageal catheter with a heat sink, according to the present disclosure.

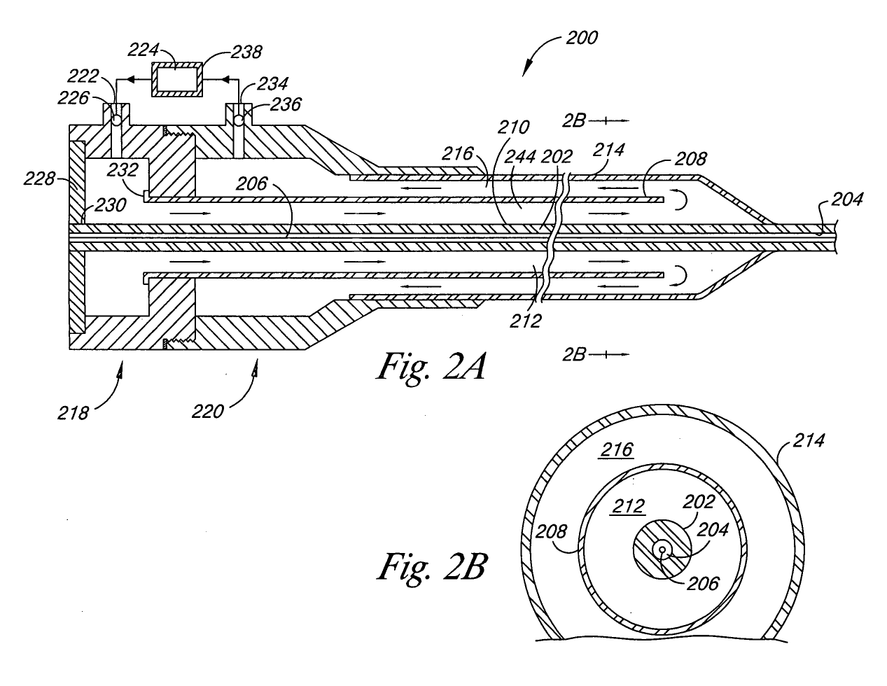

FIG. 2A illustrates a sectional view of an esophageal catheter 200 with a heat sink according to an embodiment of the present disclosure. The esophageal catheter 200 can be advanced into the esophagus, such as via the mouth or nose of the patient and performed under fluoroscopy, to a location in proximity and adjacent to the cardiac tissue to be ablated. In this embodiment, the esophageal catheter 200 includes a substantially concentric, coaxial configuration of multiple lumens or channels. The concentric, coaxial arrangement of the multiple lumens can be better understood by reference to FIG. 2B, which illustrates a cross-sectional view taken along line 2B-2B of FIG. 2A. As appreciated, lumens need not be concentric (e.g., in some instances, the lumens can be eccentrically arranged.)

In some embodiments, the esophageal catheter 200 can include an inner catheter tube 202 which defines a central conduit 204 that can receive a guide wire 206. In some embodiments, the inner catheter tube 202 may provide sufficient rigidity for insertion of the esophageal catheter 200 into the esophagus. In some embodiments, the guide wire 206, previously positioned using a guide catheter, may be required in order to facilitate advancing the esophageal catheter 200 into the esophagus.

The inner catheter tube 202 can have a single or multi-lumen construction depending on the number of channels required for a particular application. The inner catheter tube 202 may also be configured open at both ends, for example to fit over a guide wire 206, to act as a channel to inject or drain fluid, or to contain a diagnostic or therapeutic device. In some embodiments, the inner catheter tube 202 can be sealed at its distal end or configured in other ways.

In some embodiments, the esophageal catheter 200 can include an inflatable and collapsible elongate inner sleeve 208 surrounding at least a portion of the inner catheter tube 202. The inner sleeve 208 can be at least partially sealed at its distal end to an outer surface 210 of the inner catheter tube 202 so as to create an intermediate lumen 212 including an annular region with a donut-like cross section surrounding inner catheter tube 202. The annular configuration of intermediate lumen 212 is illustrated by FIG. 2B.

Also illustrated in FIGS. 2A-2B is an elongated outer sleeve 214 which is sealed at its distal end to the outer surface 210 of the inner catheter tube 202 at a point distal from the distal end of inner sleeve 208 so as to create an outer lumen 216. The outer lumen 216 can create an annular region with a donut-like cross section surrounding inner sleeve 208.

As will be appreciated, the inner sleeve 208, outer sleeve 214, and inner catheter tube 202 can be formed of a flexible material having sufficient wall strength to resist bending when moved into the esophagus. In one embodiment, suitable flexible materials include, but are not limited to, medical grade polymers and/or co-polymers, such as polypropylene, polystyrene, polyurethane, polyvinylchloride, polyethylene, polyetheretherketone, polyetherimide, polyamides, polycarbonate, biodegradables and combinations thereof. Other medical grade polymers, metals, and metal alloys are also possible. In some embodiments, the inner sleeve 208, outer sleeve 214, and inner catheter tube 202 are formed of the same material. The inner sleeve 208, outer sleeve 214, and inner catheter tube 202 can also be formed of different materials.

As discussed herein, the outer sleeve 214 can be sealed at its distal end to the outer surface 210 of the inner catheter tube 202 at a point distal from the distal end of inner sleeve 208. In some embodiments, this configuration can place the distal end of the intermediate lumen 212 in direct fluid communication with the distal end of the outer lumen 216. Although the intermediate and outer lumens 212, 216 are shown in FIG. 2A as single lumens, one or both of these lumens may be fabricated as a multi-lumen structures.

In some embodiments, the esophageal catheter 200 includes a first manifold section 218 and a second manifold section 220. The distal end of the first manifold 218 can be adapted to sealingly mate with the proximal end of the second manifold 220. For example, the distal end of the first manifold 218 can mate with the proximal end of the second manifold 220 using male and female threaded elements, in combination with a resilient O-ring. In some embodiments, the first and second manifolds 218, 220 can be adhesively bonded to one another.

In some embodiments, the first manifold 218 can include a fluid inlet port 222 connected to a source 224, such as a reservoir, of heat transfer fluid via a fluid fitting, which can also contain an inlet valve 226 and an end seal 228. The end seal 228 of the first manifold 218 can also include a centrally-located bore 230 sized to receive the inner catheter tube 202. In some embodiments, the bore 230 can be sized so as to receive both the inner catheter tube 202 and the inner sleeve 208.

In addition, in some embodiments, the proximal end of the inner sleeve 208 can include an annular lip or flange 232 projecting radially outward and capable of being bonded or sealed to an inner wall of the first manifold 218 to prevent fluid leakage between the first manifold 218 and inner sleeve 208.

In some embodiments, the second manifold 220 can include an outlet port 234, which can include an outlet valve 236. In some embodiments, the second manifold opening can be sized to receive the inner catheter tube 202, the inner sleeve 208, and the outer sleeve 214, while leaving an open annular region defined by the outside surface of the inner sleeve 208 and the inside surface of the outer sleeve 214 through which fluid can pass.

In some embodiments, the outside of the proximal end of the outer sleeve 214 can be adhesively bonded to the wall of the opening. Thus, after the distal portion of the esophageal catheter 200 is positioned in the body, fresh heat transfer fluid, at a desired temperature, can enter the first manifold 218 through the inlet port 222 (as illustrated by the fluid direction arrows), and pass through the interior cavity of the first manifold 218 into the proximal end of the inner sleeve 208 at the lip 232. The heat transfer fluid can then pass through the intermediate lumen 212 to the distal end of the inner sleeve 208, directly into the outer lumen 216, through the outer lumen 216 to the proximal end of the outer sleeve 214, and into the interior of the second manifold 220 from which it exits through the outlet port 234. The heat transfer fluid can move through the intermediate lumen 212 and the outer lumen 216 to absorb heat generated by an ablation catheter that is being used concurrently to ablate cardiac tissue.

In some embodiments, the spent heat transfer fluid exiting through the outlet port 234 may be recovered and heated or cooled (as necessary), for example, with a heating or cooling jacket 238, surrounding the source 224 of heat transfer fluid, to restore the heat transfer fluid to the desired temperature and then recycled back to the inlet port 222.

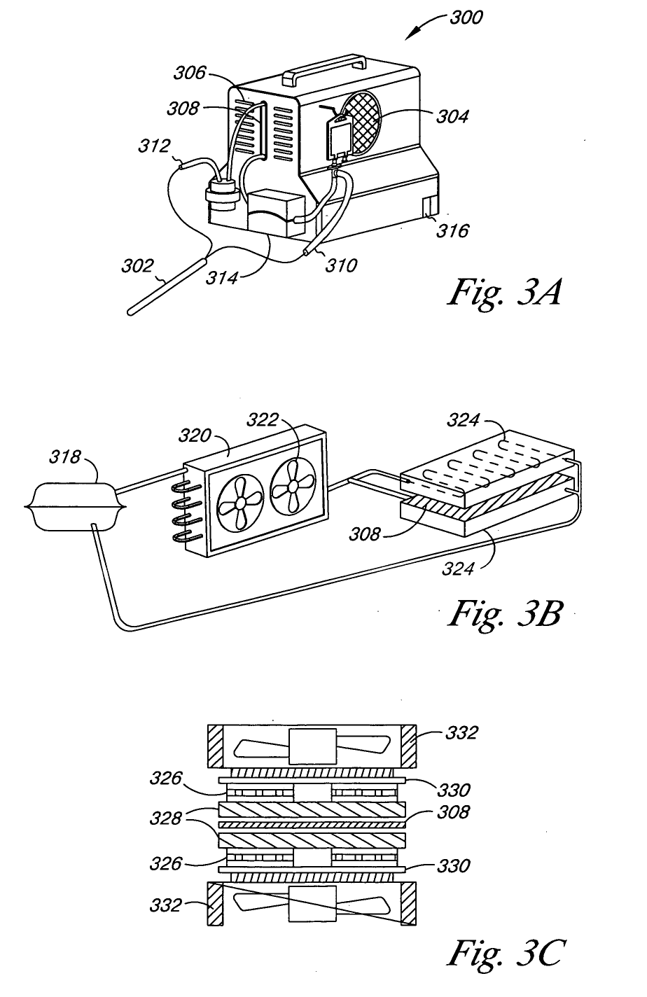

FIG. 3A is an illustration of a cooler 300 that can be used to cool the heat transfer fluid circulated through the esophageal catheter according to an embodiment of the present disclosure. In some embodiments, the cooler 300 can be used instead of having a source of heat transfer fluid that is finite, or a source of heat transfer fluid that can be cooled using a cooling jacket, as discussed herein with respect to FIG. 2A.

FIG. 3A shows a system that includes an esophageal catheter 302 with a heat sink that is in fluid communication with the cooler 300. While the term “cooler” is used herein, it is to be understood that the cooler 300 can in some embodiments warm heat transfer fluid as well as remove heat from the heat transfer fluid.

As shown in FIG. 3A, the cooler 300 can include a light weight portable plastic or metal housing that can include a handle. In some embodiments, an air intake 304 can be provided on the housing, as well as an air exhaust vent 306. In some embodiments, a heat exchange element 308, as discussed herein, can be received in the housing. As illustrated, an inlet tube 310 and an outlet tube 312 can be connected to the heat exchange element 308, and the inlet and outlet tubes 310, 312 can be connected to the esophageal catheter 302. In some embodiments, heat transfer fluid can circulate in a closed loop through the esophageal catheter 302, as discussed herein, and heat exchange element 308 with the heat transfer fluid being cooled as it passes through the heat exchange element 308.

In some embodiments, the inlet tube 310 can include a quick connect tubing pump 314 such as a peristaltic pump or diaphragm pump that can receive the inlet tube 310 and engage the inlet tube 310 externally to pump fluid there through.

In some embodiments, the housing of the portable cooler 300 can contain or otherwise support a power source 316 for the cooler 300, powering both the heat exchange element 308 and the pump 314.

FIG. 3B illustrates heat exchange components that can be included within the housing of the cooler 300. In some embodiments, a Rankin cycle compressor 318 can compress refrigerant, such as Freon, and send the Freon to a condenser 320 with cooling fans 322, which can receive air through the air intake 304 and exhaust air through the air exhaust vent 306, as illustrated in FIG. 3A.

In some embodiments, from the condenser 320 the Freon can flow through Freon lines to two heat exchange plates 324. The heat exchange plates 324 can be formed of a metal or metal-alloy, including copper, steel, or other metal. The heat exchange element 308 can be sandwiched between the plates 324 in thermal contact therewith to cool the heat exchange element 308. After passing through the heat exchange plates 324, the Freon can be sent back to the compressor 318.

FIG. 3C illustrates an embodiment where the cooler 300 can hold thermoelectric coolers (TEC) 326 that are thermally coupled with heat exchange cold plates 328 that sandwich the heat exchange element 308 and that can consequently cool the heat exchange element 308. Opposite the cold plates 328, the TEC 326 can be coupled to heat sink plates 330, which may include cooling fins. In addition, axial cooling fans 332 can remove heat from the heat sink plates 330. In some embodiments, the fans 332 can receive air through the air intake 304 and exhaust air through the air exhaust vent 306.

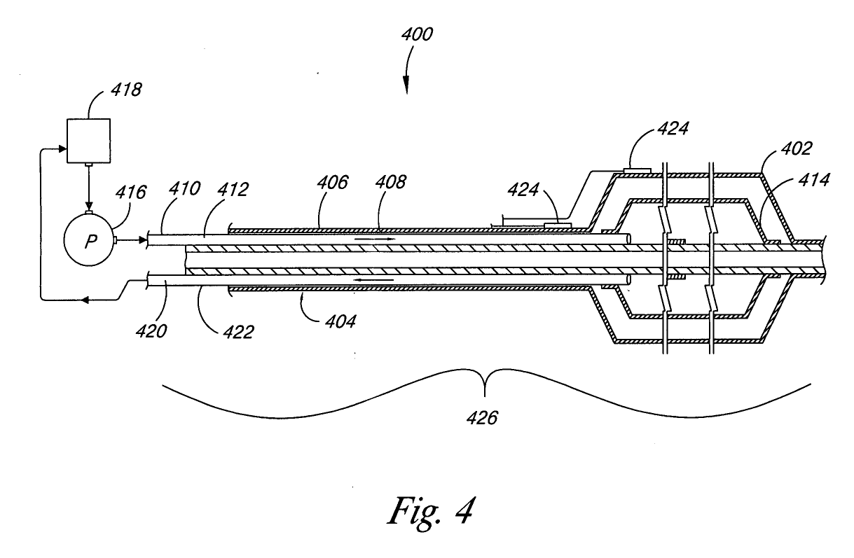

FIG. 4 illustrates a cross-sectional view of a distal segment 426 of an embodiment of an esophageal catheter 400 including a balloon 402 according to embodiments of the present disclosure. The esophageal catheter 400 includes an elongate member 404 having a proximal end and a distal end, with an outer tube 406 which defines an inflation lumen 408 in fluid communication with the interior of the balloon 402. The elongate member 404 also can include a fluid intake tube 410 that defines an intake lumen 412 in fluid communication with a chamber 414 disposed within the balloon 402. In some embodiments, the elongate member 404 and the fluid intake tube 410 can be formed of a flexible material having sufficient wall strength to resist bending when moved into the esophagus, as discussed herein.

As discussed herein, atrial ablation can cause a rise in temperature in the esophageal tissue. By operating an esophageal catheter 400 with a heat sink in the esophagus while an ablation catheter is being operated, a sufficient amount of heat can be removed to better protect the esophageal tissue from damage. In some embodiments, the temperature in the esophageal tissue can be maintained at a predetermined temperature either slightly above body temperature, at normal body temperature, or below normal body temperature. In some embodiments, once the esophageal catheter 400 is advanced into the esophagus, the balloon 402 can be dilated by forcing fluid into the balloon 402 through the inflation lumen 408. Heat transfer fluid can then be released into the chamber 414 via the fluid inlet tube 410 from a source 418 through a pump 416, or from a pressurized container, to maintain a predetermined temperature of the esophageal tissue. The heat transfer fluid can be discharged from the chamber 414 through an exhaust or drain lumen 420 defined by an outlet tube 422. The heat transfer fluid may be collected for recirculation after heat exchange, recycling, or disposal as desired.

In some embodiments, the inflation fluid used to dilate the balloon 402 can also be used as a heat sink by using an inflation fluid with a low freezing point such as an ethanol mixture. In addition, in some embodiments, the heat transfer fluid can be liquid nitrogen (N₂), Freon, nitrous oxide (N₂O) gas, and/or carbon dioxide (CO₂) gas. Other heat transfer fluids can also be used such as cold saline solution, Fluisol, or a mixture of saline solution and ethanol. Other heat transfer fluids can also be used.

In some embodiments, the esophageal catheter 400 can include the balloon 402 without the separate chamber 414. In such embodiments, the heat transfer fluid can be released into the balloon 402 through the fluid inlet tube 410 to dilate the balloon 402. The heat transfer fluid can also be discharged from the balloon 402 through the drain lumen 420 defined by the outlet tube 422 after the balloon 402 is dilated. In some embodiments, the balloon 402 can hold the heat transfer fluid for a predetermined amount of time before the heat transfer fluid is exchanged with new heat transfer fluid.

In some embodiments, the balloon 402 can also be used to hold the esophageal catheter 400 in a predetermined position in the esophagus. Once the esophageal catheter 400 is advanced into the esophagus and is positioned adjacent the ablation site, the balloon 402 can be inflated using the inflation fluid to hold the esophageal catheter 400 in place in the esophagus. In some embodiments, the esophageal catheter 400 can include a separate balloon to hold the esophageal catheter 400 in a predetermined position in the esophagus.

In some embodiments, temperature can be monitored using a feedback system including thermo-resistive sensors 424. The feedback system can be used to adjust the flow rate of the heat transfer fluid into the esophageal catheter 400 to raise or lower the temperature of the esophageal tissue based on the temperature recorded by the thermo-resistive sensors 424 and on the desired temperature of the esophageal tissue.

In some embodiments, the esophageal catheter 400 can be operated with the use of a cooler, as described herein with respect to FIGS. 3A-3B.

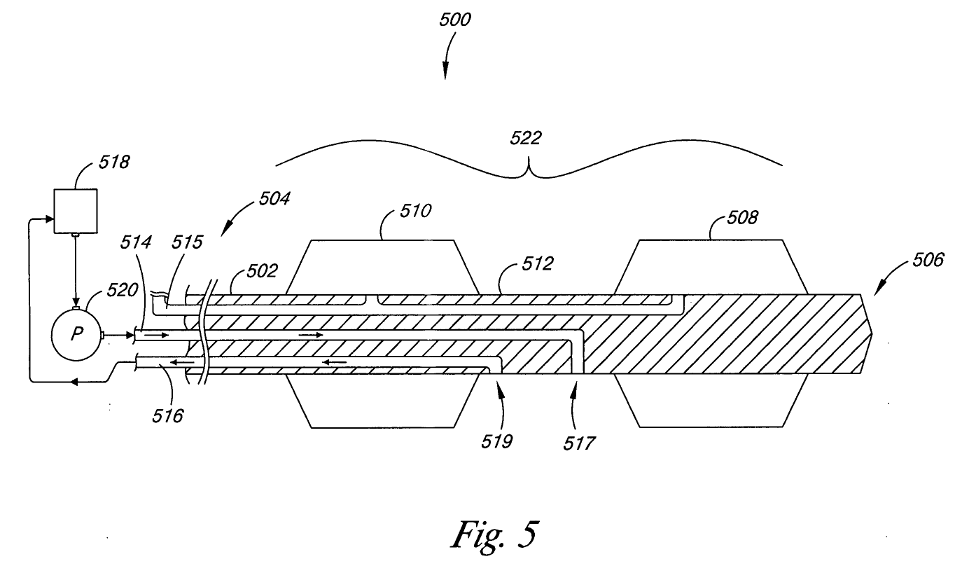

FIG. 5 illustrates an esophageal catheter 500 including two balloons according to an embodiment of the present disclosure. The esophageal catheter 500 illustrated in FIG. 5 includes an elongate member 502 with a proximal end 504 and a distal end 506. As discussed herein, the elongate member 502 can be formed of a flexible material having sufficient wall strength to resist bending when moved into the esophagus. In one embodiment, suitable flexible materials include, but are not limited to, medical grade polymers and/or co-polymers, such as polypropylene, polystyrene, polyurethane, polyvinylchloride, polyethylene, polyetheretherketone, polyetherimide, polyamides, polycarbonate, biodegradables and combinations thereof. Other medical grade polymers, metals, and metal alloys are also possible.

The elongate member 502 includes a first balloon 508 and a second balloon 510 positioned around separate portions of an exterior surface 512 of a distal segment 522 of the elongate member 502. When the esophageal catheter 500 is placed in the esophagus, the first and second balloons 508, 510 can be inflated via an inflation lumen 515. In some embodiments, the first and second balloons 508, 510 can be used to hold the esophageal catheter 500 in place and to act as a barrier for flowing heat transfer fluid, as discussed herein.

In some embodiments, the elongate member 502 can define an inlet lumen 514 extending from the proximal end 504 of the elongate member 502 to an exterior surface 512 of the elongate member 502 at a first point 517 proximal the first balloon 508. The elongate member 502 can also define an exhaust lumen 516 extending from the exterior surface 512 of the elongate member 502 at a second point 519 distal the second balloon 510 to the proximal end 504 of the elongate member 502.

In some embodiments, once the first and second balloons 508, 510 are inflated, a heat transfer fluid can flow from a coolant source 518 through a pump 520, to the inlet lumen 514. The heat transfer fluid can then flow through the inlet lumen 514 to the exterior surface 512 of the elongate member 502 at the first point 517. The heat transfer fluid can then flow between the first balloon 508 and second balloon 510, outside of the exterior surface 512 of the elongate member 502, acting as a heat sink by absorbing thermal energy from the esophageal tissue. The absorbed thermal energy in the form of warmed heat transfer fluid can then be exhausted from the space between the first and second balloons 508, 510 at the second point 519 via the exhaust lumen 516.

In some embodiments, the inflation lumen 515 can transport cooled inflation fluid to the first and second balloons 508, 510 to act as an additional heat sink to cool esophageal tissue.

In some embodiments, the heat transfer fluid can flow through the exhaust lumen 516 back to the coolant source 518, where it can be collected and cooled using a heat exchanger, as discussed herein, or discarded. As discussed herein, the esophageal catheter 500 can be coupled to a cooler (e.g., FIGS. 2A-2C) to maintain a predetermined temperature of the esophageal tissue. In other embodiments, the coolant source 518 can include a cooling jacket to cool the heat transfer fluid as it returns from the exhaust lumen 516.

As discussed herein, the heat transfer fluid can be selected from a group including, but not limited to, saline, water, and ethanol, or other swallowable fluids. In addition, the heat transfer fluid can be a low freezing point fluid such as an ethanol mixture. In addition, in some embodiments, the heat transfer fluid can be liquid nitrogen (N₂), Freon, nitrous oxide (N₂O) gas, and/or carbon dioxide (CO₂) gas.

In some embodiments, the first and second balloons 508, 510 can act as a heat sink while also securing the esophageal catheter 500 in the esophagus. The esophageal catheter 500 can be advanced into the esophagus before the first and second balloons 508, 510 are inflated with heat transfer fluid. Once the esophageal catheter 500 is in a position in the esophagus that is adjacent the ablation site, the first and second balloons 508, 510 can be inflated to a predetermined geometry where the first and second balloons 508, 510 can hold the esophageal catheter 500 in position while also acting as a heat sink.

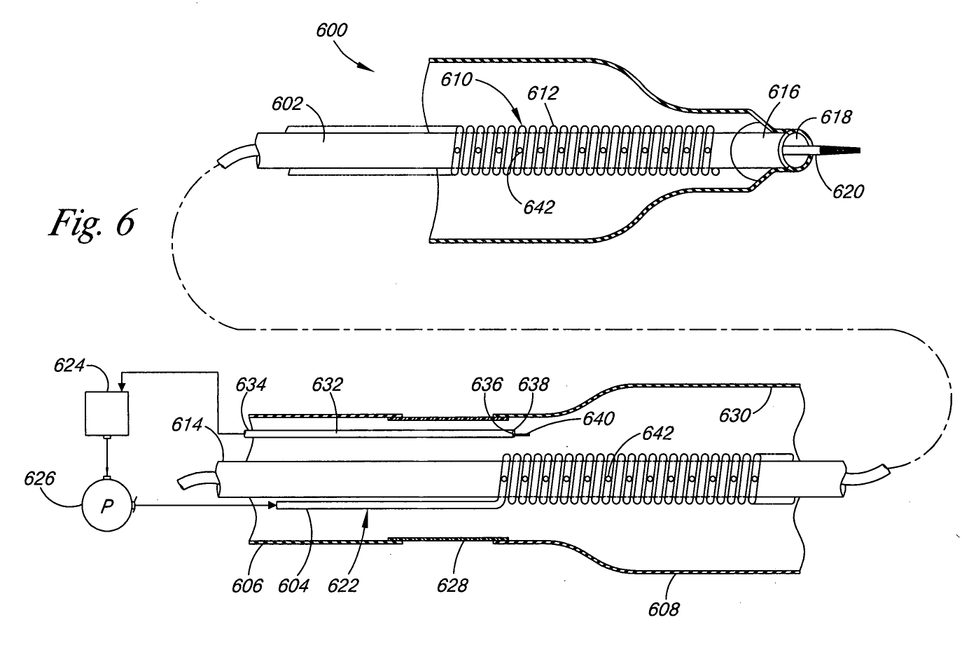

FIG. 6 illustrates a cross-sectional view of an esophageal catheter 600 according to an embodiment of the present disclosure. The esophageal catheter 600 can include an elongate member 602, a cooling tube 604 disposed adjacent to the elongate member 602, an outer tube 606 disposed over at least a portion of the cooling tube 604, and a cooling member 608 disposed over at least a portion of the cooling tube 604. In addition, cooling tube 604 may include a distal region 610 including a coil 612 disposed about the elongate member 602.

In some embodiments, the elongate member 602 can be formed of a metallic hypotube having a proximal end 614, a distal end 616, and a lumen 618 extending therethrough. The elongate member 602 can be configured and adapted to be slidably disposed over a core wire 620. In this embodiment, the elongate member 602 can be shifted in position relative to the core wire 620. This may be useful for altering the site of heat exchange while allowing the core wire 620 to remain stationary. In some embodiments, the core wire 620 can be a guidewire, tube, or other suitable structure.

The coil 612 can be slidably disposed about elongate member 602. In some embodiments, the coil 612 can be slidably disposed along essentially the entire length of the elongate member 602. Alternatively, the coil 612 can be slidable along a portion of the length of the elongate member 602 (e.g., along all or a portion of the length of the cooling member 608). Although the coil 612 is shown to be configured co-axially relative to the elongate member 602, it can be appreciated that the coil 612 could also be configured parallel to the elongate member 602 or otherwise disposed within the cooling member 608.

The cooling tube 604 may also include, in addition to distal coil 612, a proximal region 622 that may be generally straight and follow the longitudinal axis of the elongate member 602. In addition, the cooling tube 604 can be coupled to a heat transfer fluid source 624 through a pump 626, where the heat transfer fluid can be delivered to the cooling tube 604 using the pump 626. As discussed herein, the heat transfer fluid source. 624 can use a cooling jacket to cool the heat transfer fluid, or a cooler as described herein with reference to FIGS. 3A-3C.

In some embodiments, a portion of the coil 612 can be comprised of radiopaque materials. A radiopaque material is understood to be capable of producing a relatively bright image on a fluoroscopy screen or another imaging technique during a medical procedure. This relatively bright image aids the user of esophageal catheter 600 in determining the location thereof. Radiopaque materials can include, but are not limited to, gold, platinum, tungsten alloy, and plastic material loaded with a radiopaque filler. The coil 612, for example, may be at least partially gold-plated. In addition, the esophageal catheter 600 may also include additional radiopaque markers.

The outer tube 606 may be disposed over at least a portion of the cooling tube 604 near the proximal region 622. In some embodiments, the outer tube 606 may be metallic, polymeric, or a composite thereof. For example, the outer tube 606 may be formed of polyimide.

The cooling member 608 may be disposed over at least a portion of the cooling tube 604 near the distal region 610. The cooling member 608 can be coupled to the outer tube 606. Alternatively, the outer tube 606 may be coupled to a distal shaft 628. According to this embodiment, the distal shaft 628 may, in turn, be coupled to the outer tube 606. In various embodiments, the cooling member 608 may have different lengths. For example, the length of the cooling member 608 may span a portion of the length of the elongate member 602 or be comparable in size to typical angioplasty balloons.

In some embodiments, the cooling member 608 may comprise a LEAP II balloon. LEAP II balloons are comprised of polyether block amide (PEBA). Polyether block amide is commercially available from Atochem Polymers of Birdsboro, Pa., under the trade name PEBAX. Regardless of what material the cooling member 608 is formed of, the cooling member 608 may be used by allowing the coil 612 to spray heat transfer fluid onto an inner surface 630 of the cooling member 608. The cooling member 608 can then be used as a heat sink in the esophagus to protect the esophageal tissue during ablation, as discussed herein.

The esophageal catheter 600 can also include a tube 632 having a proximal end 634, a distal end 636, and a lumen 638 extending there through. The lumen 638 may be a lumen that may be used to drain heat exchange fluid from cooling member 608 if the temperature therein rises above a predetermined point. According to this embodiment, the tube 632 may further include a temperature sensor 640 that may be used to quantify the temperature proximate the cooling member 608.

In some embodiments, the esophageal catheter 600 can include a second outer tube that defines an annular lumen between the second outer tube and the outer tube 606. In such embodiments, the second outer tube can increase the strength of the esophageal catheter 600, enhance the safety of the esophageal catheter 600, and/or alter or enhance the cooling ability of the esophageal catheter 600.

As illustrated in FIG. 6, the coil 612 includes at least one opening 642. Heat exchange fluid passing through cooling tube 604 may pass through the opening 642 and be sprayed onto the inner surface 630 of the cooling member 608. Multiple embodiments of the coil 612 may include differing configurations and numbers of openings 642. Alterations in the number of openings or configuration of opening may be made, for example, the coil 612 may include 2, 4, 6, or 8 openings arranged in a generally circular arrangement such that heat exchange fluid may be sprayed as a circular ring onto the inner surface 630. Spacing between the openings 642 may be regular (i.e., 60° separation between 6 openings 642, 45° separation between 8 openings 642, etc.) or may be irregular.

In addition, the shape of the openings 642 may be configured to allow heat exchange fluid to be uniformly sprayed onto the inner surface 630. For example, the openings 642 may be configured to have a frusto-conical shape in order to uniformly spray heat exchange fluid. Alternatively, the openings 642 may have a flow directing nozzle disposed therein or be configured to have a flow directing nozzle shape in order to impart uniform coolant spray.

In use, the esophageal catheter 600 can be advanced into the esophagus near where ablation is to occur. Heat exchange fluid can then be released through the openings 642 of the cooling tube 604. In some embodiments, the heat exchange fluid can include cold water or cold saline solution, among others.

The openings 642 within the coil 612 can be manufactured in a number of different ways. For example, the openings 642 can be drilled using a cobalt puncture bit. Other ways of forming the openings 642 are also possible.

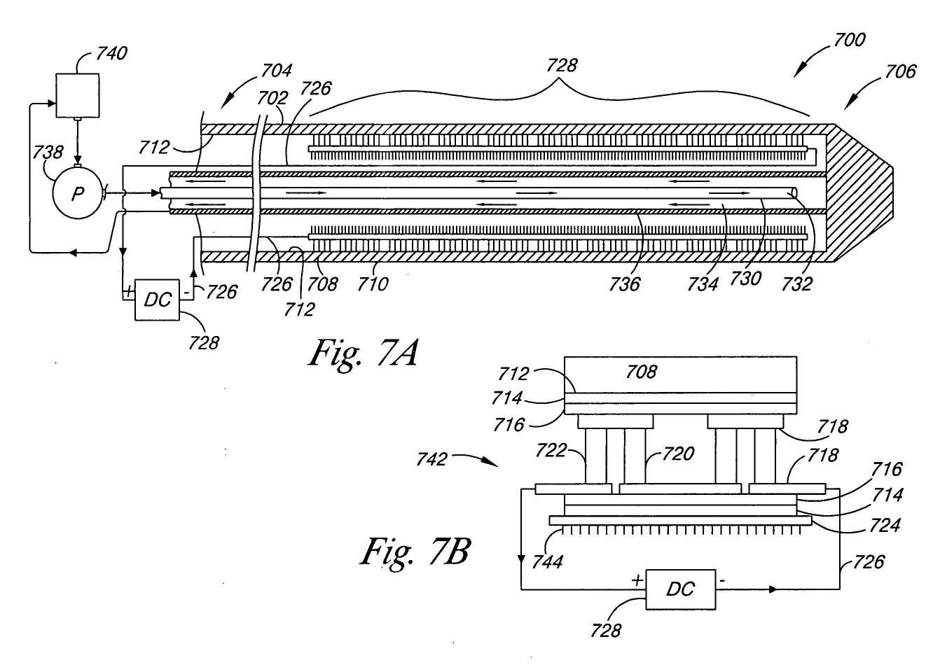

FIG. 7A illustrates an esophageal catheter 700 according to an embodiment of the present disclosure. In the embodiment illustrated in FIG. 7A the esophageal catheter 700 includes a heat sink in the form of a thermo-electric cooler (TEC). FIG. 7B illustrates a close up view of an embodiment of a TEC according to the present disclosure. A TEC exploits the Peltier effect to cause heat to flow between fused, dissimilar metal surfaces when subjected to a DC current. When the DC current passes through a TEC device, heat is absorbed at one side and released to the opposite side during operation.

The embodiment of the esophageal catheter 700 illustrated in FIG. 7A includes an elongate member 702 with a proximal end 704 and a distal end 706, where a distal segment 728 of the elongate member 702 includes the TEC. In some embodiments, the elongate member 702 can include an outer catheter tube 708 with an outside surface 710, where the outer catheter tube 708 at the distal segment 728 is cooled when a DC current is applied to a TEC coupled to an inside surface 712 of the outer catheter tube 708.

As discussed herein, FIG. 7B illustrates an embodiment of TEC 742. In the embodiment illustrated in FIG. 7B, the TEC consists of a cold plate, i.e., the outer catheter tube 708, a thermal interface 714, a ceramic plate 716, a trace 718, a first metal 720, a second metal 722, a heat plate 724, and a conductor 726 to carry current to the trace 718 from a DC power source 728. In some embodiments, the thermal interface 714 can be coupled to the inside surface 712 of the outer catheter tube 708. The thermal interface 714 can consist of grease, a pad, or solder. In addition, the trace 718 can be formed of a thermally conductive material, for example, aluminum or copper. Other materials for the trace 718 are also possible.

In some embodiments, the first and second metal 720, 722 can be formed of several different metal pairs. The first and second metal 720, 722 can be chosen from a group including, but not limited to, iron-constantan, copper-nickel, bismuth-telluride, or lead-constantan. In some embodiments, the first and second metal 720, 722 are an n-doped semiconductor and a p-doped semiconductor.

In some embodiments, the TEC can require that the heat transferred from the cold plate, i.e., the outer catheter tube 708 is carried away from the heat plate 724. In such embodiments, the heat plate 724 can include a heat sink in the form of fins 744 extending from the heat plate 724. In this embodiment, once the heat generated by the heat plate 724 has traveled to the ends of the fins 744, the air surrounding the fins 744 can be heated. The density of the air can then change due to the increase in temperature, causing air that is less dense to rise while air that is more dense sinks. In this way, the air flow caused by the change in density can cool the heat plate 724 by convection. Other fluids are also possible.

In addition, in some embodiments, the esophageal catheter 700 can include an inner catheter tube 730 extending from the proximal end 704 of the elongate member 702 to the distal end of the TEC. In some embodiments, the inner catheter tube 730 can define a central conduit 732 to carry heat transfer fluid from the proximal end 704 to the distal end 706 of the elongate member 702. For example, cooled air can be forced from the proximal end 704 to the distal end 706 of the elongate member 702. In this example, the use of cooled air can allow the esophageal catheter 700 to be small enough to be swallowed without the use of anesthetics. In some embodiments, the esophageal catheter 700 can be coated with Novocain to prevent a gag reflex.

In some embodiments, the esophageal catheter 700 can include an intermediate sleeve 736 extending from the proximal end 704 of the elongate member 702 to the distal tip of the elongate member 702, effectively sealing the TEC from the heat transfer fluid delivered via the inner catheter tube 730. The heat transfer fluid can then flow from the proximal end to the distal end of the inner catheter tube 730 (indicated by arrows) and back to the proximal end 704 of the elongate member by flowing through an intermediate lumen 734 defined by the outer surface of the inner catheter tube 730 and the inside surface of the intermediate sleeve 736.

In some embodiments, the heat transfer fluid can be delivered to the inner catheter tube 730 using a pump 738 and a heat transfer fluid source 740. As discussed herein, the heat transfer fluid source 740 can use a cooling jacket to cool the heat transfer fluid, or a cooler as described herein with reference to FIGS. 3A-3C.

As discussed herein, tissue ablation can be preceded by three-dimensional (3-D) mapping of the conductive pathways and sources of cardiac tissue. In some embodiments, a transesophageal echocardiogram (TEE) probe can be used do the mapping. An ultrasonic TEE probe is a gastroscope with an ultrasonic transducer at the distal tip. The tip can be inserted down the esophagus and sometimes into the stomach from which the transducer scans the heart for diagnostic imaging or monitoring purposes. The transducer is moved while in the body by a short articulation section preceding the tip which is controlled from a handle outside the body. A diagnostic TEE probe produces cardiac images which do not have to contend with the chest wall, ribs, or lungs as transthoracic probes do. Rather than having to transmit and receive ultrasound from between the ribs, the TEE probe has direct access to the heart from the esophagus or stomach, unimpeded by the ribs.

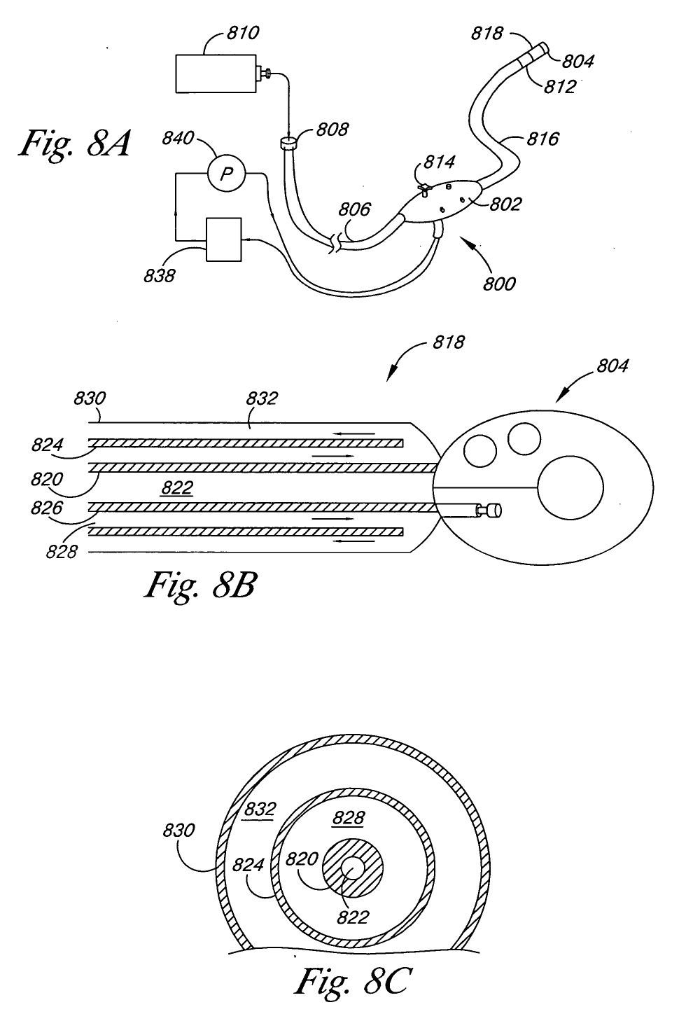

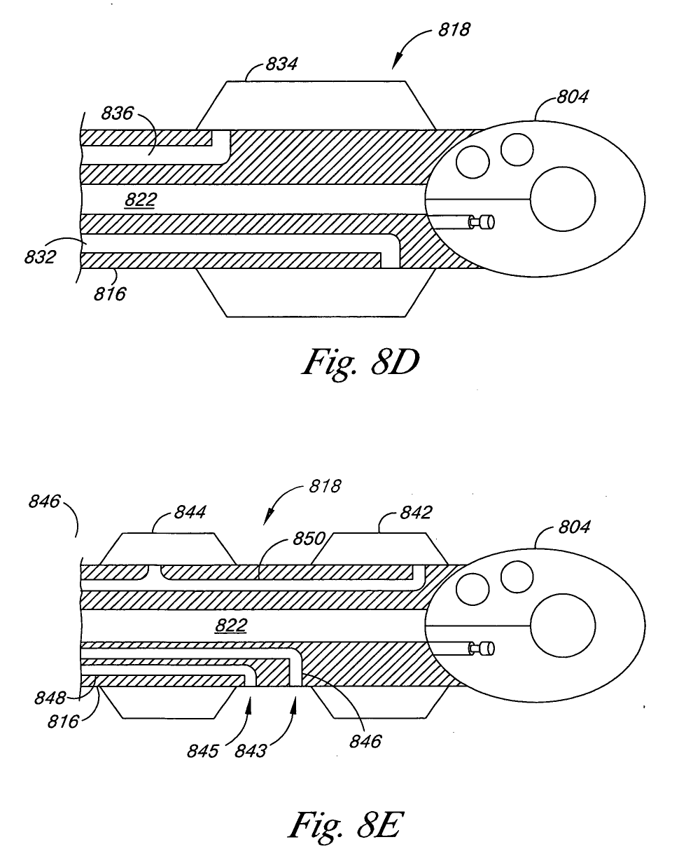

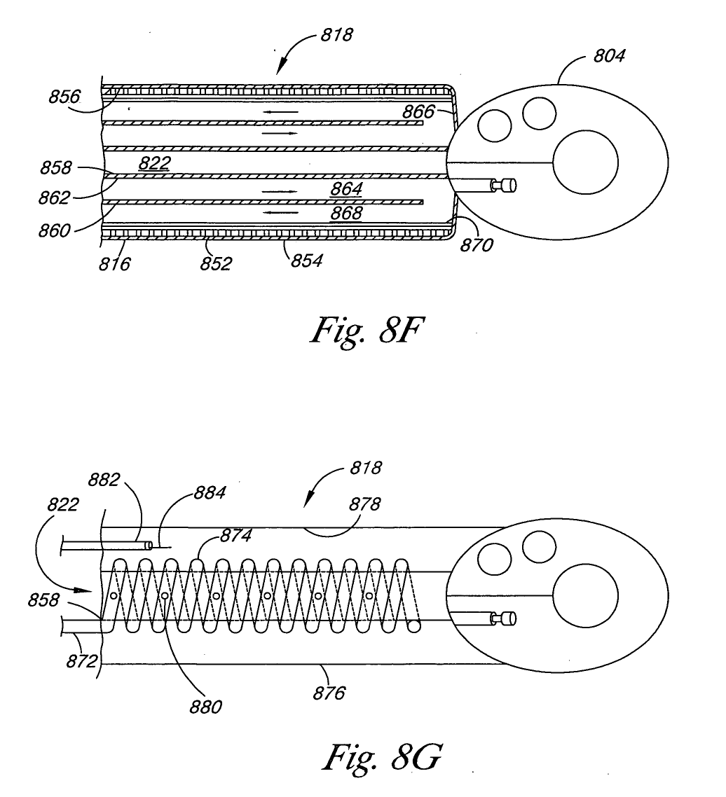

FIG. 8A illustrates a transesophageal echocardiogram (TEE) probe 800 according to an embodiment of the present disclosure. In some embodiments, the TEE probe 800 can include a proximal handle 802 where the controls of the probe 800 are located and a distal tip portion 804 where an ultrasonic transducer is located. The ultrasonic transducer can be connected to an ultrasound system 810 by extending a cable 806 through an elongate member 816 from the distal tip portion 804, through the proximal handle 802, and to a connector 808. In some embodiments, the connector 808 can be suitable for connecting the probe 800 to the ultrasound system 810 which can energize the probe 800 and display images formed from the acoustic signals transmitted and received by the transducer contained in the distal tip portion 804. In some embodiments, the distal tip portion 804 can be deflected for proper positioning of the transducer by bending a flexible section 812. This deflection can be produced by rotation of wheel 814 which is mechanically coupled to section 812 by cables or other mechanisms which extend through the elongate member 816.

The TEE probe 800 can also include a heat sink 818 on the elongate member 816 adjacent the distal tip portion 804. By including a heat sink 818, the TEE probe 800 can be advanced into the esophagus and used to display images formed from the acoustic signals transmitted and received by the transducer while also maintaining a predetermined temperature in the esophagus while an ablation catheter, as discussed herein, is ablating tissue.

In some embodiments, the heat sink 818 can be separate from the TEE probe 800 and can be slidably positioned adjacent the distal tip portion 804 of the TEE probe 800 as a heat sink 818 jacket. In such embodiments, the heat sink 818 jacket could slide onto the probe 818 to act as a heat sink, while not affecting the TEE probe 800 operation. Alternatively, the heat sink 818 can be a constructed part of the TEE probe 800. Also, in some embodiments, the heat sink 818 or the TEE probe 800 can include radiopaque markers to identify the placement of the TEE probe 800 while in the esophagus.

FIG. 8B illustrates a detailed view of an embodiment of the distal tip portion 804 of the TEE probe 800 and the heat sink 818 in the form of a cooling jacket on the outside surface of the elongate member adjacent the distal tip portion 804 according to the present disclosure. In this embodiment, the cooling jacket can be similar to a heat exchange catheter, as described herein with respect to FIGS. 2A-2B. Specifically, in some embodiments, the cooling jacket can include a substantially concentric, coaxial arrangement of multiple lumens or channels.

As illustrated in FIG. 8B, the TEE probe 800 can include an inner catheter tube 820 which defines a central conduit 822 for receiving the cables and/or other mechanisms that control the movement of the elongate member 816. The inner catheter tube 820 can have a single or multi-lumen construction depending on the number of channels required for a particular application. In some embodiments, the inner catheter tube 820 can be sealed at its distal end to prevent heat transfer fluid from entering the central conduit 822.

In the embodiment illustrated in FIGS. 8B-8C, the cooling jacket can also include an inflatable and collapsible elongated inner sleeve 824 surrounding at least a portion of the length of the inner catheter tube 820. The inner sleeve 824 can be at least partially sealed at its distal end to an outer surface 826 of the inner catheter tube 820 so as to create an intermediate lumen 828 including an annular region with a donut-like cross section surrounding inner catheter tube 820. The annular configuration of intermediate lumen 828 is illustrated by FIG. 8C.

Also illustrated in FIGS. 8B-8C is an elongated outer sleeve 830 which is sealed at its distal end to the outer surface 826 of the inner catheter tube 820 at a point distal from the distal end of inner sleeve 824 so as to create an outer lumen 832. Also, the outer sleeve 830 can be sealed to the outer surface 826 of the inner catheter tube 830 at a point adjacent the distal tip portion 804 of the TEE probe 800. The outer lumen 832 can create an annular region with a donut-like cross section surrounding inner sleeve 824.

As will be appreciated, the inner sleeve 824, outer sleeve 830, and inner catheter tube 820 can be formed of a flexible material having sufficient flexibility to allow the flexible section 812 of the elongate member 816 to deflect. In one embodiment, suitable flexible materials include, but are not limited to, medical grade polymers and/or co-polymers, such as polypropylene, polystyrene, polyurethane, polyvinylchloride, polyethylene, polyetheretherketone, polyetherimide, polyamides, polycarbonate, biodegradables and combinations thereof. Other medical grade polymers, metals, and metal alloys are also possible. In some embodiments, the inner sleeve 824, outer sleeve 830, and inner catheter tube 820 are formed of the same material. The inner sleeve 824, outer sleeve 830, and inner catheter tube 820 can also be formed of different materials.

As discussed herein with respect to FIGS. 2A-2B, the heat sink 818 in the form of a cooling jacket can include a first manifold section and a second manifold section that include inlet and outlet ports to deliver heat transfer fluid to the cooling jacket from a coolant source. In some embodiments, heat transfer fluid can pass through the inlet port into the proximal end of the inner sleeve 824, through the intermediate lumen 828 to the distal end of the inner sleeve 824, directly into the outer lumen 832, and to the proximal end of the outer sleeve 830, exiting the cooling jacket through the outlet port. In addition, the cooling jacket can use a heat transfer fluid that is cooled using a cooler, as discussed herein with respect to FIGS. 3A-3C.

FIG. 8D illustrates a cross sectional view of an embodiment of the distal tip portion 804 of the TEE probe with a cooling jacket on the elongate member 816 adjacent the distal tip portion 804. In the embodiment illustrated in FIG. 8D, the elongate member 816 defines a central conduit 822 to receive the cables and/or other mechanisms that control the movement of the elongate member 816. In some embodiments, the cooling jacket can be formed of an inflatable balloon 834 coupled to the outside surface of the elongate member 816 adjacent the distal tip portion 804. The elongate member 816 can define a fluid inlet lumen 836, separate from the central conduit 822, extending from the proximal end of the elongate member 816 to a proximal end of the balloon 834. In some embodiments, the elongate member 816 can define an outlet lumen 832 extending from the proximal end of the elongate member 816 to a distal end of the balloon 834. In some embodiments, the outlet lumen 832 and the central conduit are formed such that the outlet lumen 832 and central conduit 822 are separate.

In some embodiments, a heat transfer fluid can be delivered via a heat transfer fluid source 838 and pump 840, as discussed herein, for example, with respect to FIG. 8A, through the inlet lumen 836 to the balloon 834. Once the balloon 834 is inflated, the heat transfer fluid can exit the balloon 834 via the outlet lumen 832 to be cooled in a cooler (e.g., FIG. 2A-2C) or discarded, as discussed herein.

FIG. 8E illustrates a cross sectional view of an embodiment of the distal tip portion 804 of the TEE probe 800 with a cooling jacket on the outer surface of the elongate member 816 adjacent the distal tip portion 804. In the embodiment illustrated in FIG. 8E, the elongate member 816 defines a central conduit 822 to receive the cables and/or other mechanisms that control the movement of the elongate member 816, as discussed herein. In some embodiments, the cooling jacket can be formed of a first balloon 842 and a second balloon 844 coupled to the exterior surface of the elongate member 816 adjacent the distal tip portion 804. The first and second balloons 842, 844 can act as a cooling jacket when a heat transfer fluid flows between the first and second balloons 842, 844 to maintain a predetermined temperature in the esophagus.

In some embodiments, the elongate member 816 can define an inlet lumen 846 extending from the proximal end of the elongate member 816 to a the exterior surface of the elongate member at a first point 843 proximal the first balloon 842. The elongate member 816 can also define an exhaust lumen 850 extending from the exterior surface at a second point 845 distal the second balloon 844 to the proximal end of the elongate member 816. The elongate member 816 can also define an inflation lumen 850 extending from the proximal end of the elongate member 816 to the first and second balloons 842, 844.

In some embodiments, an inflation fluid can flow through the inflation lumen 850 to inflate the first and second balloons 842, 844. Once the balloons 842, 844 are inflated, a heat transfer fluid can flow from a coolant source 838 through a pump 840 to the inlet lumen 846, through the inlet lumen 846 to the first point, through the exterior surface of the elongate member 816. The heat transfer fluid can flow outside the elongate member 816 to the second point 845 on the exterior surface of the elongate member 816. The heat transfer fluid can then through the second point 845 through the exhaust lumen 848 back to the coolant source 838, where it can be collected and cooled using a heat exchanger, as discussed herein, or discarded. As discussed herein, the TEE probe 800 can be coupled to a portable cooler (e.g., FIGS. 2A-2C) to maintain a predetermined temperature of the esophageal tissue. In other embodiments, the coolant source 838 can include a cooling jacket to cool the heat transfer fluid as it returns from the exhaust lumen 848.

FIG. 8F illustrates an embodiment of a TEE probe 800 including a cooling jacket according to the present disclosure. FIG. 8F illustrates a cross-sectional view of the distal tip portion 804 and a portion of the elongate member 816 adjacent the distal tip portion 804. In the embodiment illustrated in FIG. 8F, the cooling jacket can be a TEE positioned adjacent the distal tip portion 804, as discussed herein with respect to FIGS. 6A-6B.

The embodiment of the TEE probe 800 illustrated in FIG. 8F includes a elongate member 816 including an outer catheter tube 852 with an outside surface 854, where the outer catheter tube 852 is cooled when a DC current is applied to a TEC coupled to an inside surface 856 of the outer catheter tube 852. As discussed herein, in some embodiments, the TEC heat plate can be cooled by convection. Alternatively, the TEC heat plate can be cooled by the flow of a heat transfer fluid that will not affect the operation of the TEC if the TEC comes into contact with the heat transfer fluid, for example, forced air.

In some embodiments, the TEE probe 800 can include an inner catheter tube 858 defining a central conduit 822 for receiving cables and/or other mechanisms to control the movement of the flexible section 812 of the elongate member 816, as discussed herein. In some embodiments, the inner catheter tube 858 can be sealed at its distal end to prevent heat transfer fluid from entering the central conduit 822. In the embodiment illustrated in FIG. 8F, the cooling jacket can include an inflatable and collapsible elongated inner sleeve 860 surrounding at least a portion of the length of the inner catheter tube 858. The inner sleeve 860 can be at least partially sealed at its distal end to an outer surface 862 of the inner catheter tube 858 so as to create an intermediate lumen 864 including an annular region with a donut-like cross section surrounding inner catheter tube 858.

Also illustrated in FIGS. 8F is an elongated outer sleeve 870 which is sealed at its distal end to the inner surface 866 of the outer catheter tube 852 at a point distal from the distal end of inner sleeve 860 so as to create an outer lumen 868. The outer lumen 868 can create an annular region with a donut-like cross section surrounding inner sleeve 860.

As will be appreciated, the inner sleeve 860, outer sleeve 870, and inner catheter tube 858 can be formed of a flexible material having sufficient flexibility to allow the flexible section 818 of the elongate member 816 to deflect, as discussed herein. Also, as discussed herein with respect to FIGS. 2A-2B, the cooling jacket can include a first manifold section and a second manifold section that include inlet and outlet ports to deliver heat transfer fluid to the cooling jacket from a coolant source. In some embodiments, heat transfer fluid can pass through the inlet port into the proximal end of the inner sleeve 860, through the intermediate lumen 864 to the distal end of the inner sleeve 860, directly into the outer lumen 868, and to the proximal end of the outer sleeve 870, exiting the cooling jacket through the outlet port. In addition, the cooling jacket can use a heat transfer fluid that is cooled using a cooler, as discussed herein with respect to FIGS. 3A-3C.

As discussed herein, in some embodiments, the TEE probe 800 can include an inner catheter tube 858 defining a central conduit 822 for receiving cables and/or other mechanisms to control the movement of the flexible section 812 of the elongate member 816. In some embodiments, the inner catheter tube 858 can be sealed at its distal end to prevent heat transfer fluid from entering the central conduit 822. In the embodiment illustrated in FIG. 8G, the cooling jacket can include a cooling tube 872 with a distal region including a coil 874 disposed about the inner catheter tube 858, and a cooling member 876 disposed about at least a portion of the cooling tube 872,

The coil 874 can include at least one opening 880 where heat transfer fluid can be sprayed from the coil 874 onto an inner surface 878 of the cooling member 876 when heat transfer fluid is supplied to the cooling tube 872 from a heat transfer fluid source. In some embodiments, the cooling member 876 may be formed of a LEAP II balloon, as discussed herein, as well as other materials. In addition, the cooling jacket can use a heat transfer fluid that is cooled using a cooler, as discussed herein with respect to FIGS. 3A-3C.

The cooling jacket can also include a tube 882 to drain heat exchange fluid from cooling member 876 if the temperature therein rises above a predetermined point and/or after a predetermined amount of time. In addition, the tube 882 can further include a temperature sensor 884 that may be used to quantify the temperature proximate the cooling member 876.

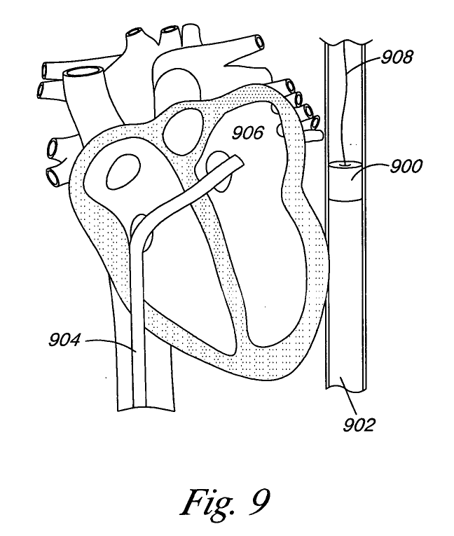

FIG. 9 illustrates an additional embodiment of an esophageal protection device according to the present disclosure. The esophageal protecting device includes an esophageal heat sink 900 inside an esophagus 902 while an ablation catheter 904 is directing energy towards a cardiac tissue site 906. The esophageal heat sink can be coupled to a wire 908 to hold the esophageal heat sink 900 in the esophagus 902 near the cardiac tissue site 906.

The esophageal heat sink 900 can be formed of a material having a sufficiently high heat capacity to enable the ablation catheter 904 to direct energy towards a cardiac tissue site 906 while absorbing enough thermal energy to maintain the esophageal tissue at a predetermined temperature. In some embodiments, the esophageal heat sink 900 can be formed of a solid, such as a metal. A metal esophageal heat sink 900 can be formed of aluminum, magnesium, nickel, or titanium, among others. In some embodiments, the esophageal heat sink 900 can be formed of frozen water. In some embodiments, the esophageal heat sink 900 can be a liquid sealed in a biocompatible esophageal membrane, for example, the esophageal heat sink can be formed of water, milk, ethanol, sodium chloride, potassium hydrate, or ammonia, among others.

In some embodiments, the esophageal heat sink 900 and the wire 908 can be formed of a bioadsorbable material. As used herein, “bioadsorbable” refers to a material that can be adsorbed and/or digested by the body of the patient. In such embodiments, once the ablation procedure is completed, the wire 908 can simply be cut and the esophageal heat sink 900 can be allowed to pass into the stomach of the patient.

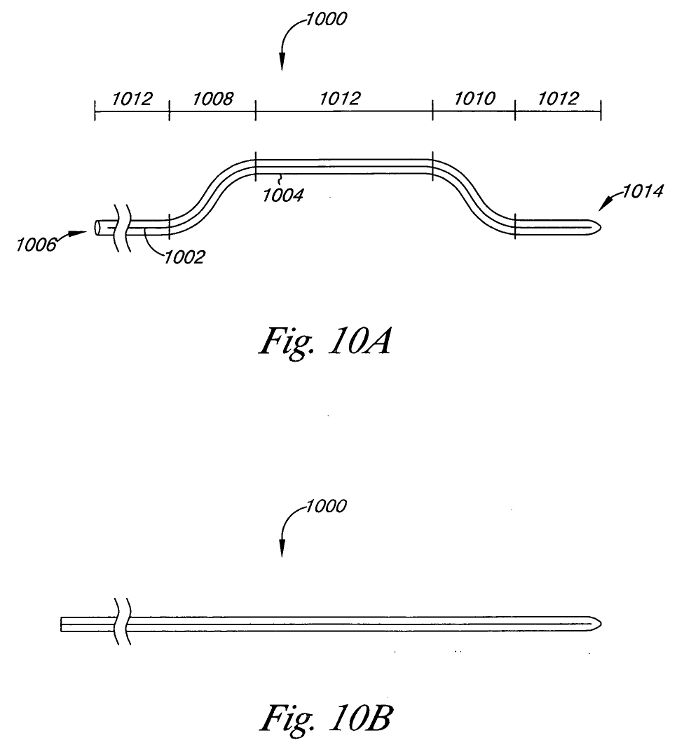

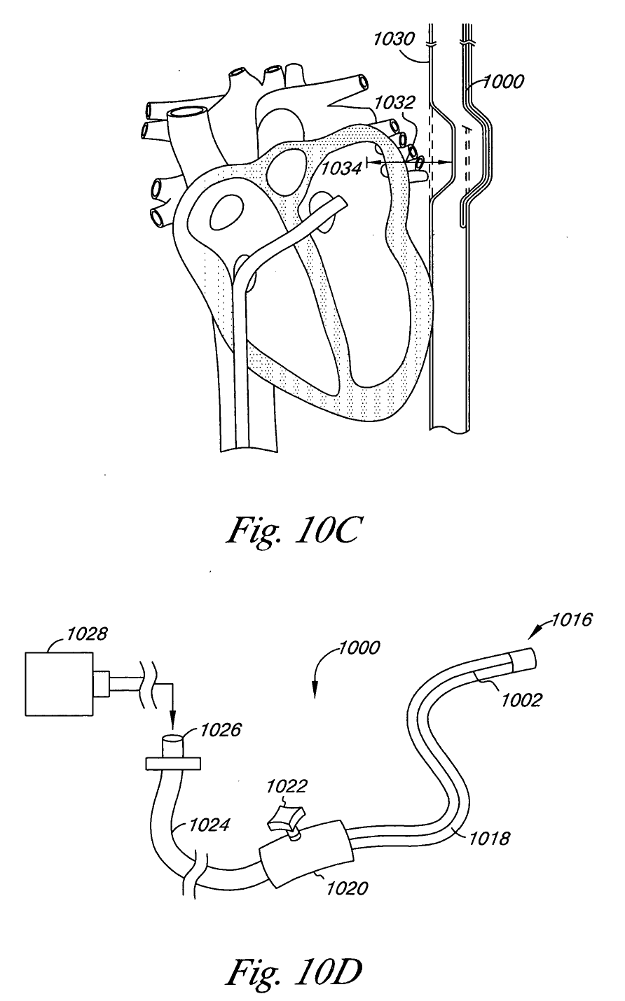

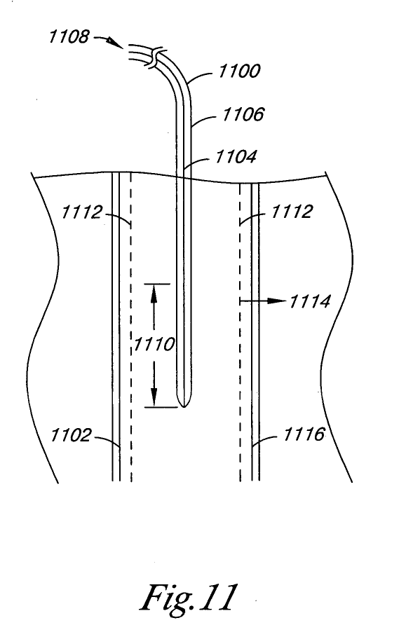

Embodiments of the present disclosure also provide for mechanical manipulation of the esophagus to minimize the esophagus' exposure to ablation energy delivered to the heart. For example, FIGS. 10A-10B illustrate an embodiment of an esophageal stylet 1000 according to the present disclosure. In some embodiments, an esophageal stylet 1000 can be used to move the esophagus a distance away from the cardiac tissue site to prevent heat from being transferred into the esophageal tissue during an ablation procedure. In one embodiment the esophageal stylet 1000 can be formed of an elongate member 1002 having a pre-determined shape. In some embodiments, the esophageal stylet 1000 can include a catheter 1004, where the elongate member 1002 is placed inside a catheter lumen 1006, and the catheter 1004 is be formed of a soft plastic material that can prevent injury to the esophagus.

In some embodiments, the esophageal stylet 1000 can have a first configuration, illustrated in FIG. 10B, and a second configuration, illustrated in FIG. 10A. Other shapes for the second configuration are also possible, including an arc, a square, or a triangular shape, among others. In such embodiments, the esophageal stylet 1000 can be advanced into the esophagus while in the first configuration. Once the esophageal stylet 1000 is in a position in the esophagus, the esophageal stylet 1000 can be manipulated into the second configuration to aid in moving the esophagus a distance away from an ablation site.

FIG. 10C illustrates the esophageal stylet 1000, while in the second configuration, when it is placed in the esophagus 1030. As illustrated, the esophageal stylet 1000 can move the esophagus a distance 1032 away from the ablation site 1034. In some embodiments, by increasing the distance 1032 between the ablation site 1034 and the esophageal tissue, the heat generated by an ablation catheter, as discussed herein, is less likely to damage the esophageal tissue than when the esophagus 1030 is not manipulated away from the ablation site 1034.

Referring to FIG. 10A, the esophageal stylet 1000 can change from the first configuration to a second configuration in various ways. In one embodiment, the catheter 1004 can be formed of a polymeric material where two sections 1008, 1010 of the catheter 1004 are more flexible than the other portions 1012 of the catheter 1004. In this embodiment, the elongate member 1002 can be attached to the inside surface of the distal tip 1014 of the catheter 1004 and can be pulled while holding the catheter 1004 in place in the esophagus. The elongate member 1002 can be attached to the inside surface of the distal tip 1014 of the catheter by an adhesive, by forming the elongate member 1002 and the catheter 1004 at the same time from the same material, or by welding the elongate member 1002 to the distal tip 1014 when the catheter is formed of a metal material, among others.

In some embodiments, by pulling the elongate member 1002, the two flexible sections of the catheter 1004 can bend into the second configuration (e.g., FIG. 10A). In addition, in some embodiments, the elongate member 1002 can be locked into the pulled position to enable the catheter 1004 to be moved without having to hold the elongate member 1002 in a fixed position.

In some embodiments, the elongate member 1002 can be formed of a shape-memory alloy. In such embodiments, the elongate member 1002 can be advanced into the esophagus in the first configuration (e.g., FIG. 10B). Once the esophageal stylet 1000 is in the esophagus, a current can be applied to the elongate member 1002 to force the elongate member 1002 into the second configuration (e.g., FIG. 10A). In some embodiments, the elongate member 1002 can be formed of Nitinol. The elongate member 1002 can also be formed of other shape-memory alloys including, but not limited to, CuZnAl, or CuAlNi, among others.

As will be appreciated, the catheter 1004 can be formed of a flexible material having sufficient flexibility to allow the elongate member 1002 to shift from the first configuration to the second configuration. Suitable flexible materials include, but are not limited to, medical grade polymers and/or co-polymers, such as polypropylene, polystyrene, polyurethane, polyvinylchloride, polyethylene, polyetheretherketone, polyetherimide, polyamides, polycarbonate, biodegradables and combinations thereof. Other medical grade polymers, metals, and metal alloys are also possible.

In some embodiments, the esophageal stylet 1000 can be used to move the esophagus away from an ablation site by moving the esophageal stylet 1000 while in the second configuration. To determine what direction the esophagus should be moved, a patient can perform a barium swallow while undergoing videofluorography or X-ray analysis.

FIG. 10D illustrates an embodiment of an esophageal stylet 1000 incorporated into a transesophageal echocardiogram (TEE) probe, as discussed herein. As discussed herein, the TEE probe can include an ultrasonic transducer at the distal tip 1016 of a elongate member 1018 and a proximal handle 1020 where the controls of the probe are located. In some embodiments, the elongate member 1002 can extend from the handle 1020 to the distal tip 1016 of the elongate member 1018, and the rotation of a wheel 1022 can cause the elongate member 1002 to change from the first configuration to a second configuration, as discussed herein. In addition, in some embodiments, the TEE probe can include a cable 1024 which extends from the handle 1020 and terminates at a connector 1026 used to connect the probe to an ultrasound system 1028 which energizes the probe and displays images formed from the acoustic signals transmitted and received by the transducer. Also, the cable 1024 can carry a current to the elongate member 1002 when the elongate member 1002 is formed of a shape-memory alloy to shift the elongate member 1002 from the first configuration to the second configuration, as discussed herein. The TEE probe can also include other features, as discussed herein.

FIG. 11 illustrates an embodiment of an esophageal stylet 1100 according to the present disclosure when the esophageal stylet 1100 is inside an esophagus 1102. The esophageal stylet 1100, as discussed herein, can include an elongate member 1104 inside a lumen 1108 of catheter 1106. As discussed herein, the catheter 1106 can be formed of a soft polymer material that can be inserted into the esophagus 1102 without damaging the esophageal tissue.

In some embodiments, a distal segment 1110 of the esophageal stylet 1100 can be formed of a magnetic material and coupled to the elongate member 1104. The magnetic distal segment 1110 can be coupled to the elongate member 1104 by crimping, laser welding, or chemical adhesion, among other ways. When positioned in an esophagus, the esophageal stylet 1100 can be used to move the esophagus 1102 away from an ablation site to prevent an increase in temperature of the esophageal tissue. In some embodiments, a magnetic field 1112 can be applied to the magnetic distal segment 1110 in a selected orientation, and an approximately transverse magnetic gradient 1114 can be applied to the magnetic distal end 1110 to draw the esophageal stylet 1100 against the surface 1116 of the body structure (e.g., the esophagus).

The magnetic field 1112 and gradient 1114 can be simultaneously applied with a single permanent magnet, a pair of permanent magnets, a single electromagnetic coil, or a pair of electromagnetic coils. In addition, the magnetic distal end 1104 can be moved in another direction by changing the direction of the applied magnetic field 1106. This can be done by moving or changing the orientation of the source of the applied magnetic field 1106 and gradient 1108. Also, as discussed herein, the esophagus 1102 position can be determined by performing a barium swallow while undergoing videofluorography or X-ray analysis.

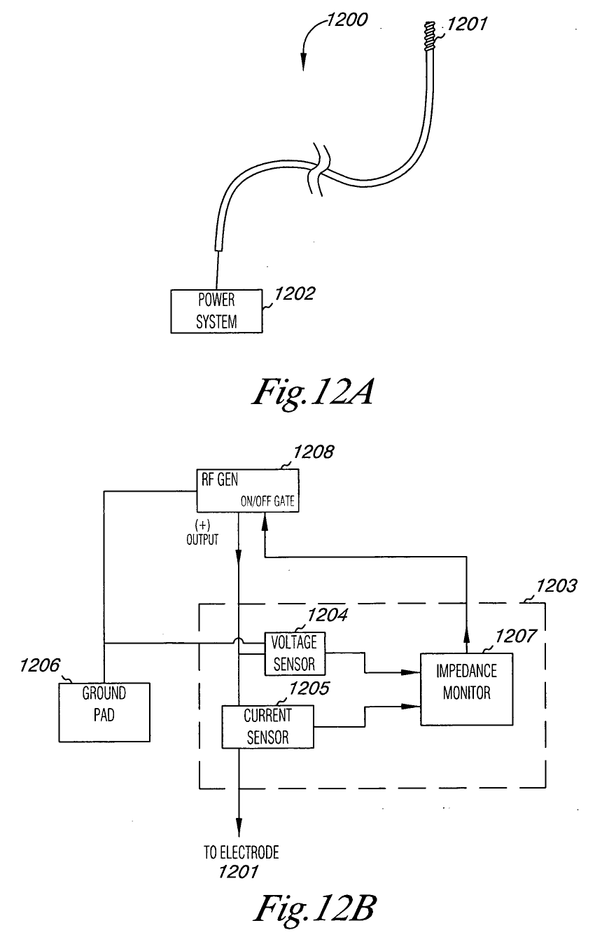

FIG. 12A illustrates an esophageal catheter 1200 including an electrode 1201 according to an embodiment of the present disclosure. In some embodiments, an esophageal catheter 1200 including an electrode 1201 can be advanced into an esophagus while an ablation catheter is directing energy towards a cardiac tissue site. The electrode 1201 can be used to measure the electrical impedance of the esophageal tissue. The esophageal catheter 1200 can be coupled to a power system 1202.

FIG. 12B illustrates an embodiment of the power system according to the present disclosure. In some embodiments, the power system 1202 can include a feedback monitor 1203 which includes a voltage sensor 1204 and a current sensor 1205. Both the voltage sensor 1204 and current sensor 1205 can each be connected directly to an impedance monitor 1207. Further, the impedance monitor 1207 can be connected via a line to the on/off gate of the RF generator 1208. Further, the voltage sensor 1204 is connected to the RF generator 1208, to the electrode 1201 through the current sensor 1205, and to the ground pad 1206. As so connected, the voltage sensor 1204 provides a voltage input to the impedance monitor 1207. The current sensor 1205 meanwhile, is connected between the RF generator 1208 and the electrode 1201 to provide a current input to the impedance monitor 1207. With these inputs, the impedance monitor 1207 provides a close-loop control for the device by activating the on/off gate of the RF generator 1208 to discontinue the transmission of RF energy from the electrode 1201 whenever a predetermined impedance is measured between the electrode 1201 and the ground pad 1206.

In some embodiments, the feedback monitor 1203 can include a switch between the current sensor 1205 and the electrode 1201. In such embodiments, instead of activating an on/off switch in the RF generator 1208, the impedance monitor 1207 activates the switch between the current sensor 1205 and the electrode 1201 for the same purpose. Other places for a switch are also possible.

As discussed herein, radiopaque materials and/or radiopaque markers can be used with the embodiments described herein to determine the location of the esophageal catheter. A radiopaque material is understood to be capable of producing a relatively bright image on a fluoroscopy screen or another imaging technique during a medical procedure. This relatively bright image aids the user of esophageal catheter in determining the location thereof. Radiopaque materials can include, but are not limited to, gold, platinum, tungsten alloy, and plastic material loaded with a radiopaque filler. The esophageal catheter, for example, may be at least partially gold-plated. In addition, the esophageal catheter may also include additional radiopaque markers.

While the present invention has been shown and described in detail above, it will be clear to the person skilled in the art that changes and modifications may be made without departing from the spirit and scope of the invention. As such, that which is set forth in the foregoing description and accompanying drawings is offered by way of illustration only and not as a limitation. The actual scope of the invention is intended to be defined by the following claims, along with the full range of equivalents to which such claims are entitled.

In addition, one of ordinary skill in the art will appreciate upon reading and understanding this disclosure that other variations for the disclosure described herein can be included within the scope of the present disclosure.

I claim:

1. An ablation system, comprising:
an esophageal catheter having a heat sink; and
an ablation catheter having at least one ablation element to deliver ablation energy, where heat generated by the ablation energy is absorbed by the heat sink.

2. The ablation system of claim 1, where the esophageal catheter includes:
an inner catheter tube defining a central conduit to receive a guidewire;
an elongate inner sleeve surrounding at least a portion of the inner catheter tube, where the inner sleeve is at least partially sealed at a distal end to an outer surface of the inner catheter tube to create an intermediate lumen; and
an elongate outer sleeve sealed to the outer surface of the inner catheter tube at a point distal from the distal end of the inner sleeve to create an outer lumen, where heat transfer fluid can move through the intermediate lumen and the outer lumen to absorb heat generated by the ablation catheter.

3. The ablation system of claim 1, where the esophageal catheter includes:
an elongate member having a proximal end and a distal end, an inflation lumen, an intake lumen, and an outtake lumen;
a balloon positioned around a portion of the elongate member and in fluid communication with the inflation lumen; and
a chamber disposed within the balloon and in fluid communication with the intake lumen and the outtake lumen, where coolant fluid can move through the intake lumen into the chamber and through the outtake lumen to absorb heat generated by the ablation catheter.

4. The ablation system of claim 1, where the esophageal catheter includes:
an elongate member having a proximal end and a distal end, an inlet lumen, an exhaust lumen, and an inflation lumen;
a first balloon and a second balloon positioned around separate portions of the elongate member, where the inflation lumen is in fluid communication with the first balloon and the second balloon, the inlet lumen extends to an exterior surface of the elongate member at a first point proximal the first balloon, and the exhaust lumen extends to the exterior surface of the elongate member at a second point distal the second balloon, where a heat transfer fluid can flow through the inlet lumen to the exterior surface of the elongate member at the first point, outside the exterior surface of the elongate member to the second point, and through the exterior surface of the elongate member through the exhaust lumen to absorb heat generated by the ablation catheter.

5. The ablation system of claim 1, where the esophageal catheter includes:
an elongate member adapted to be slidably disposed about a core wire;
a cryo tube disposed adjacent to the elongate member, the cryo tube having a proximal region and a distal region including a coil disposed about at least a portion of the elongate member, where the coil includes at least one opening;
an outer tube disposed over at least a portion of the cryo tube; and
a cooling member disposed over the coil and coupled to the outer tube.

6. The ablation system of claim 1, where the esophageal catheter includes:
an elongate member with a proximal end and a distal end, and an inside surface adjacent the distal end;
a thermoelectric cooling device coupled to the inside surface of the elongate member;
an inner catheter tube inside the elongate member, where the inner catheter defines a central conduit extending from the proximal end to the distal end of the elongate member;
an intermediate sleeve that extends inside the elongate member from the proximal end to the distal end of the elongate member and seals to the inside surface of the elongate member and a point distal the thermoelectric cooling device.

7. The ablation system of claim 6, where heat transfer fluid can move through the central conduit toward the distal end of the elongate member, and can return toward the proximal end through an intermediate lumen defined by an outside surface of the inner catheter tube and an inside surface of the intermediate sleeve.

8. The ablation system of claim 1, where the esophageal catheter includes at least one positioning member, where the positioning member has no column strength.

9. The ablation system of claim 8, where at least one positioning member is a string.

10. The ablation system of claim 10, where the heat sink and the at least one positioning member are formed from a biodegradable substance.

11. The ablation system of claim 1, where the esophageal catheter includes:
a trans-esophageal echocardiogram probe; and
a cooling jacket that surrounds at least a part of an outside surface of the esophageal echocardiogram probe.

12. The ablation system of claim 1, where the esophageal catheter includes a thermocouple adjacent the heat sink, where the thermocouple provides signals to change the heat sinks ability to absorb heat.

13. The ablation system of claim 1, where the esophageal catheter includes an inflatable member coupled to the esophageal catheter, where the inflatable member can hold the esophageal catheter in a predetermined position.

14. A method of protecting esophageal tissue, comprising:
applying ablation energy to a cardiac tissue site; and
removing heat from esophageal tissue to maintain a predetermined temperature of the esophageal tissue during the application of ablation energy

15. The method of claim 14, where maintaining the temperature of the esophageal tissue includes removing thermal energy from esophageal tissue heated as a result of applying the ablation energy.

16. The method of claim 15, where removing thermal energy from the esophageal tissue includes:
positioning a heat exchanger catheter into an esophagus; and
circulating a heat exchange fluid through the heat exchanger catheter to remove thermal energy from the esophageal tissue.

17. The method of claim 16, where removing thermal energy from the esophageal tissue includes:
positioning a trans-esophageal echocardial probe with a cooling jacket into an esophagus; and
circulating a heat exchange fluid through the cooling jacket to remove thermal energy from the esophageal tissue.

18. The method of claim 15, where removing thermal energy from the esophageal tissue includes positioning a heat sink in an esophagus.

19. The method of claim 14, where maintaining a temperature of esophageal tissue includes moving at least a portion of the esophageal tissue a distance away from the cardiac tissue site.

20. The method of claim 19, where moving at least a portion of the esophageal tissue includes:
locating the esophagus;
inserting an esophageal catheter into the esophagus; and
manipulating the esophageal catheter to move the esophagus a distance away from the cardiac tissue site.

21. The method of claim 20, where manipulating the esophageal catheter includes applying a magnetic field to the esophageal catheter to move the esophageal tissue.

22. The method of claim 14, where maintaining a temperature of the esophageal tissue includes:
inserting an electrical impedance detector into an esophagus;
detecting an impedance of a radio frequency ablation energy directed towards the cardiac tissue site; and
stopping the generation of ablation energy when the impedance detected reaches a predetermined lower limit.

23. A transesophageal echocardiogram probe, comprising:
an elongate member;
an ultrasonic transducer coupled to the elongate member, the ultrasonic transducer having a conductor to allow ultrasound signals to be sent and received from the probe;
a heat sink that surrounds at least a part of the elongate member to remove thermal energy.

24. The transesophageal echocardiogram probe of claim 23, where the heat sink includes:
an elongate inner sleeve surrounding at least a portion of an inner catheter tube;
an intermediate lumen defined by an outside surface of the inner catheter tube and an inside surface of the elongate inner sleeve; and
an elongate outer sleeve sealed at a distal end to the outer surface of the inner catheter tube at a point proximal the ultrasonic transducer, where heat transfer fluid can move through the inner catheter tube and the intermediate lumen to absorb heat.

25. The transesophageal echocardiogram probe of claim 24, where the heat sink includes:
a balloon positioned around a portion of the elongate member and in fluid communication with a fluid inlet lumen defined by the elongate member extending from the proximal end of the elongate member to a proximal end of the balloon; and
a fluid outlet lumen defined by the elongate member extending from a distal end of the balloon to the proximal end of the elongate member, where heat transfer fluid can move through the fluid inlet lumen into the balloon and through the fluid outlet lumen to absorb heat.

26. The transesophageal echocardiogram probe of claim 24, where the heat sink includes a first balloon and a second balloon positioned around separate portions of the elongate member, where an inflation lumen is in fluid communication with the first balloon and the second balloon, an inlet lumen extends to an exterior surface of the elongate member at a first point proximal the first balloon, and an exhaust lumen extends to the exterior surface of the elongate member at a second point distal the second balloon, where a heat transfer fluid can move through the inlet lumen to the exterior surface of the elongate member at the first point, outside the exterior surface of the elongate member to the second point, and through the exterior surface of the elongate member through the exhaust lumen to absorb heat generated by the ablation catheter.

27. The transesophageal echocardiogram probe of claim 24, where the heat sink includes:
a thermo-electric cooling device coupled to an inside surface of at least a portion of the elongate member;
an inner catheter tube defining a central conduit for receiving a cable to control the movement of the elongate member;
an elongate inner sleeve surrounding at least a portion of a length of the elongate member at least partially sealed at a distal end to an outer surface of the inner catheter tube creating an intermediate lumen;
an elongate inner sleeve sealed at a distal end to an inner surface of the elongate member at a point distal the thermoelectric cooling device to create an outer lumen, where the inner sleeve is in fluid communication with a heat transfer fluid source.
